(19) United States
(12) Patent Application Publication
(10) Pub. No.: US 2002/0133232 A1
(43) Pub. Date: Sep. 19, 2002

(54) Microstructured dual sided membrane for tissue growth and regeneration

(76) Inventors: John L. Ricci, Middleton, NJ (US); Harold Alexander, Short Hills, NJ (US)

Correspondence Address: Melvin K. Silverman, Suite 440, 4901 North Federal Highway, Fort Lauderdale, FL, 33308, US

(21) Appl. No.: 10/081,478

(22) Filed: Feb. 25, 2002

Related U.S. Application Data
(63) Continuation-in-part of application No. 09/500,038, filed on Feb. 8, 2000, now Pat. No. 6,419,491.
Application No. 09/500,038 is a continuation-in-part of application No. 08/996,224, filed on Dec. 22, 1997, now Pat. No. 6,147,666.
Application No. 08/996,224 is a continuation of application No. 08/639,712, filed on Apr. 29, 1996, now abandoned.
Application No. 08/639,712 is a continuation of application No. 08/390,805, filed on Feb. 15, 1995, now abandoned.
Application No. 08/390,805 is a continuation of application No. 08/146,790, filed on Nov. 2, 1993, now abandoned.

(51) Int. Cl. A61F 2/28; A61F 2/02

(52) U.S. Cl. 623/23.5; 623/23.74; 623/23.75

(57) ABSTRACT

An implantable substantially planar bioabsorbable article for the separation and regeneration of tissue at a tissue defect site includes first surface of the article, which may be circular or elliptical, that is provided with a soft tissue side intended for direction towards soft or subcutaneous tissue and having a microtextured surface optimized for promotion of ingrowth of soft tissue. An opposite side of the article comprises a different microgrooved surface, optionally including osteoconductive chemical properties, and physically oriented against or within the bone defect site toward and against the center of the defect into which any graft material placed. The membrane is flexible and of sufficient density to accommodate sutures or is circumfentially provided with perforations to hold sutures.

### REFERENCE TO RELATED APPLICATIONS

This case is a continuation-in-part of application Ser. No. 09/500,038, filed Feb. 8, 2000 which is a continuation-in-part of application Ser. No. 08/996,224, filed Dec. 22, 1997 (now abandoned) which is a continuation of application Ser. No. 08/639,712, filed Apr. 29, 1996 (now abandoned) which is a continuation of Ser. No. 08/390,805 filed Feb. 15, 1995 (now abandoned) which is a continuation of Ser. No. 08/146,790, filed Nov. 2, 1993 (now abandoned).

### BACKGROUND OF THE INVENTION

The prior art has recognized the capacity of multiple grooved surfaces and substrates to exert topographical control over cell behavior. See for example Development 108, 635-644 (1990) by Clark, et al, entitled Topographical Control of Cell Behavior: Multiple Grooved Substrata; and Journal of Biomedical Materials Research, Vol. 24, 1203-1219 (1990) by Chehroudi et al, entitled Titanium-Coated Micromachined Grooves of Different Dimensions Affect Epithelial and Connective-Tissue Cells Differently In Vivo. Two of the authors of said Development 108 article, namely, Curtis and Wilkinson, have secured U.S. Pat. No. 5,833,641 (1998) entitled Wound Healing Material, which teaches a device, for use in promoting wound healing, made of substrate formed of a biologically acceptable material having thereon means, such as grooves, capable of orienting cell growth, to enable guided tissue repair. Other means of producing micromachined grooved substrates to achieve topographical control of cell behavior are taught in U.S. Pat. No. 5,607,607 (1998) to Naiman et al, entitled System and Assemblage for Producing MicroTexturized Substrates and Implants.

Further, PTO publication WO9210218A1 (1992) to Hayes, entitled Implant able Bioabsorbable Article, which relates to an implantable bioabsorbable article for the separation and regeneration of tissue at a tissue defect site, the article comprising a fibrous matrix affixed to one surface of a cell barrier sheet material. When implanted at a surgical site, it allows ingrowth of tissue into the fibrous matrix side permitting tissue regeneration at that side while separating such area from tissue ingrowth at the opposite side of the article. As such, a teaching similar in concept to that of Curtis et al is disclosed.

The instant invention differs from the teachings of the above, as well as from the teachings of our predecessor applications (see Reference to Related Applications) in that the focus herein is that of a thin, flexible bioabsorbable article, having the external appearance of a bandage or bandaid, having particularity utility in the context of surgery at the surface of a bone and where, after such surgery, a natural interface between bone tissue and soft tissue, such as epithelial tissue, is to occur in a natural fashion. As such, no art known to the inventors teaches a dual sided membrane for guided tissue healing and regeneration in which one side thereof is particularly adapted to assist hard tissue growth while the other side thereof is particularly adapted to further soft tissue growth, and which is to further adapted to the postoperative development of a normal interface between the endogenous interface between such hard and soft tissue.

A need for such a guided tissue regeneration (hereinafter “GTR”) membrane arises in various surgical contexts including, without limitation, dentistry and orthopedics where it is often necessary, due to a given pathology or trauma to remove a damaged area of bone which is normally covered by skin, gum, or other soft tissue, and then to insert therein a graft material such as a resorbable calcium sulfate polymeric matrix. However, for such procedures to be successful, it is necessary to ensure the integrity and stability of such grafts as well as to protect the in situ site from bacterial activity. It is also necessary to assure that the re-attachment of normally enveloping soft tissue does not occur in a manner which is inconsistent with the surgical objectives of the bone repair procedure. It is, accordingly, to these ends that the instant invention is directed.

### SUMMARY OF THE INVENTION

The instant invention relates to an implantable substantially planar bioabsorbable article for the separation and regeneration of tissue at a tissue defect or wound site. A first surface of the article, which may be circular or elliptical, is provided with a soft tissue side, intended for direction towards soft or subcutaneous tissue, and having a microtextured surface optimized for promotion of ingrowth of soft tissue, while an opposite side thereof will have a different microgrooved surface, optionally including osteoconductive chemical properties, and physically oriented within the bone defect or wound site toward and against the center of the defect into which graft material may be placed. The GTR membranes is flexible and of sufficient density to accommodate sutures or are circumfentially provided with perforations to hold sutures.

It is accordingly an object of the invention to provide a dual sided GTR membrane for the purpose of assisting in a wound healing process.

It is another object to provide a substantially planar bioabsorable wound healing material having particular utility in the control or guidance of tissue regeneration during a healing process thereby encouraging the regeneration of tissue of normal function and morphology.

It is a further object of the invention to provide a GTR membrane of the above type in which respective upper and lower surfaces thereof are particularly physically and chemically optimized to establish stable interfaces with soft and hard tissue upon respective sides thereof such that, during healing, soft tissue will interfere with the normal or desired mode of healing of hard or bone tissue at an in vivo wound site.

It is a yet further object to provide a flexible, absorbable membrane that may be sutured about a wound and/or graft site to stabilize the same and to provide a temporary barrier against undesirable soft tissue ingrowth and bacterial penetration during the healing period.

It is a yet further object to control micro-topographical cell behavior during healing processes which occur at interfaces between hard and soft tissue.

The above and yet other objects and advantages of the present invention will become apparent from the hereinafter set forth Brief Description of the Drawings, Detailed Description of the Invention and claims appended herewith.

### BRIEF DESCRIPTION OF THE DRAWINGS

FIG. 1 is a perspective view showing one geometry of the inventive GTR membrane.

FIG. 2 is an enlarged view, on a scale of about 500 magnifications, of the upper or soft tissue side of the GTR membrane, showing one potential cell growth orienting geometry thereof.

FIG. 3 is a vertical cross-sectional view taken through Line 3-3 of FIG. 2.

FIG. 4 is an enlarged view, on a scale of about 500 magnifications, of the bottom or hard tissue side of the GTR membrane showing one potential cell growth orienting geometry thereof.

FIG. 5 is a vertical cross-sectional view taken through Line 5-5 of FIG. 4.

FIG. 6 is a schematic view showing a contemplated application of the instant invention at a wound site at which a graft material has been employed and at an interface between hard and soft tissue.

FIG. 7 is a transverse cross-sectional view taken along Line 7-7 of FIG. 6.

FIG. 8 is a transverse cross-sectional view taken along Line 8-8 of FIG. 6.

FIGS. 9 to 14 are top plan views of potential microgeometries of either surface of the GTR membrane.

FIGS. 15 to 22 are vertical cross-sectional views of possible GTR membrane surface geometries.

### DETAILED DESCRIPTION OF THE INVENTION

For purposes of the present disclosure, the term “wound,” is to be understood in a broad sense as including wounds occurring as a result of accident, surgery or dentistry, or in relation to defects caused by disease. A wound will generally comprise a discontinuity in an existing tissue, regardless of the cause thereof.

It is generally advantageous that any article or substrate, to achieve the objectives of the present invention, degrade and eventually disappear from within and about the wound site during or after completion of the healing process, so that the substrate itself does not occupy space that should otherwise be filled with cells, i.e., that the device not interfere with the process of wound healing. Toward this end, it has been found desirable that a biodegradable substrate should degrade completely within a period of three to nine weeks, although this may depend on the severity of the wound and the speed of healing of a particular wound type. It may also be dependent upon the physiology of the particular patient. The above timeframe is in distinction to earlier views expressed in the prior art to the effect that such a biodegradable substrate should degrade completely within two to fourteen days. See for example Curtis, U.S. Pat. No. 5,833,641 supra. Accordingly, the instant invention, in addition to its other distinctions over the art, contemplates a period of absorbability well in excess of, and beyond, that earlier believed to be optimal for wound healing materials and articles. The inventors have found that this objective can be achieved in two ways, the first by increasing the thickness of the substrate over the teaching of the prior art and, secondly, by increasing the density or concentration of the membrane material. For example, said reference to Curtis teaches a preferable range of 50-100 microns for the thickness of a sheet-like substrate, while the instant invention contemplates a membrane of 200-500 microns.

A wide variety of biodegradable, biologically acceptable materials are known in the art which may serve as a substrate for the material of the instant GTR membrane. Many of these materials are polymeric and include materials such as polylactic acid homopolymers, polyglycollic acid copolymers, combinations thereof, polylactones, polypeptides, polyvinyl alcohols and natural polymers such as collagen and polysaccharides. In the case of homopolymers, corresponding copolymers with other such materials may also be employed.

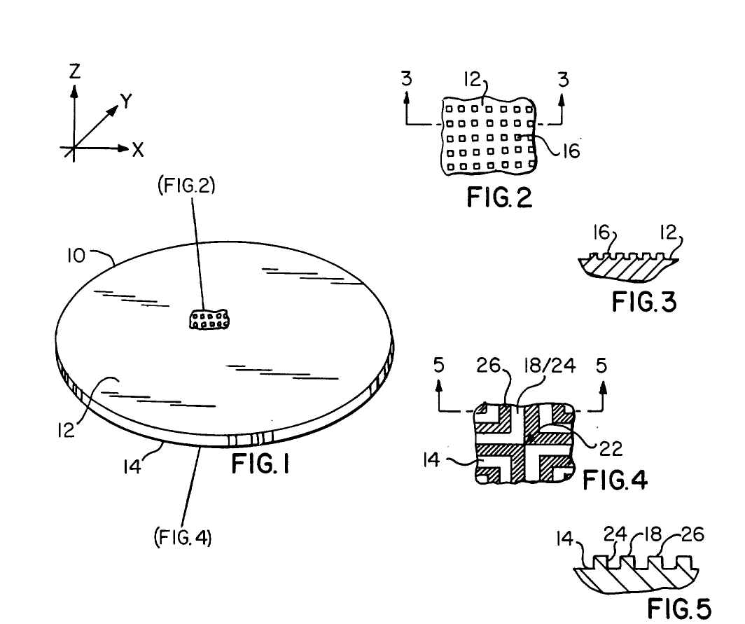

In one embodiment of the invention, namely, that shown in FIG. 1, the diameter of the substantially circular GTR membrane is that of about 12 millimeters (slightly less than 0.5 inches) and, as above noted, will have a width in a range of 200 to 500 microns (0.2 to 0.5 millimeters).

Said GTR membrane 10 includes an upper or soft tissue side 12 and a lower or hard tissue side 14 (see FIG. 1). Research over the last ten years (see Reference to Related Applications) has indicated that certain microtextured surfaces, and certain specific geometries thereof are more effective in the establishment of a stable soft tissue interface than are others. More particularly, as is shown in the enlarged magnified schematic views of FIGS. 2, 3 13 and 17, it is believed that a so-called micro-post surface having six micron grooves, post widths and post heights will establish a stable interface between the GTR membrane 10 and soft tissue that surface 12 is contacted with. It is therein to be appreciated that the specific micro-texture or geometry of surface 12 will be considerably dictated by the type of soft tissue of interest. However, in general, the function of micro-posts 16 is that of effecting a cytophobic separation between posts 16 that corresponds to the morphology of individual cells or small groups thereof, thereby permitting the efficient integration of the micro posts into a normal healing or tissue regeneration pattern of the soft tissue itself. In generally, cells or soft tissue such as cells of the gum and epithelium has been found to fall in a range of 1 to 8 microns. As such, this range would generally dictate the dimensionality of said grooves, post widths and post heights shown in FIGS. 2 and 3. Also, by defining such cytophobic regions for specific tissue, undesirable other tissue is excluded from interface with soft tissue side 12 of the GTR membrane 10.

With respect to the bone or hard tissue side 14 of the membrane 10, said surface typically includes osteocondictive chemical properties in addition to the microtexturing of surface below discussed. Appropriate such osteocondictive surfaces may include a composite of polymeric and micro- or nano-particulate hydroxyapatite to form an appropriate base upon which to construct microtexturing 16.

In general, most wounds or bone defects 19 to be repaired will have a definable center 20. See FIGS. 6 to 8. It is accordingly a strategy in the design of the instant GTR membrane to provide a corresponding center point 22 (see FIGS. 4 and 6) of side 18 to which the geometry of channels 24 is directed to encourage a maximum ingrowth of bone tissue towards center 20 of the wound or bone defect. In a preferred embodiment, it has been determined that channels 24 may have a primary dimension of 12 microns as the width and height of the microstructure 18 with separations 26 of similar dimensions therebetween. Such a 12-micron dimension is reflective of our research of many years which indicate that oseoblast cells are generally of greater size and are epithelial and muscular cells. Accordingly, a greater dimensionality of the microtextruring of lower side 14 will be more appropriate relative to that of said upper side 12, this typically in a range of 10 to 25 microns.

The inventive GTR membranes 10, as it would appear in vivo is shown in the views of FIGS. 6 through 8. More particularly, there is shown gum or epetilial tissue 28, bone tissue 30, and a bone defect or wound area 19 into which has been placed a graft material 32. Between soft tissue 28 and bone tissue 30 is placed the inventive membrane 10 to effect micro topographical control of cell behavior of the respective hard and soft tissue. More particularly, through the function of membrane 10, bone tissue 30 and the wound/graft area 19/32 is effectively compartmentalized from soft tissue 28 thereby precluding undesirable ingrowths of soft tissue to the bone and related bacterial action which typically originates from soft tissue, particularly, in dental procedures. Accordingly, the wound/graft area 19/32 is effectively isolated as to structure, cell morphology, and bacterial action, this for the contemplated period of healing typically, as above noted, a three to nine week period during which membrane 10 will gradually biodegrade into the surrounding tissue.

It is also to be appreciated that membrane 28 may also serve as a spacer and surgical packing means in many procedures.

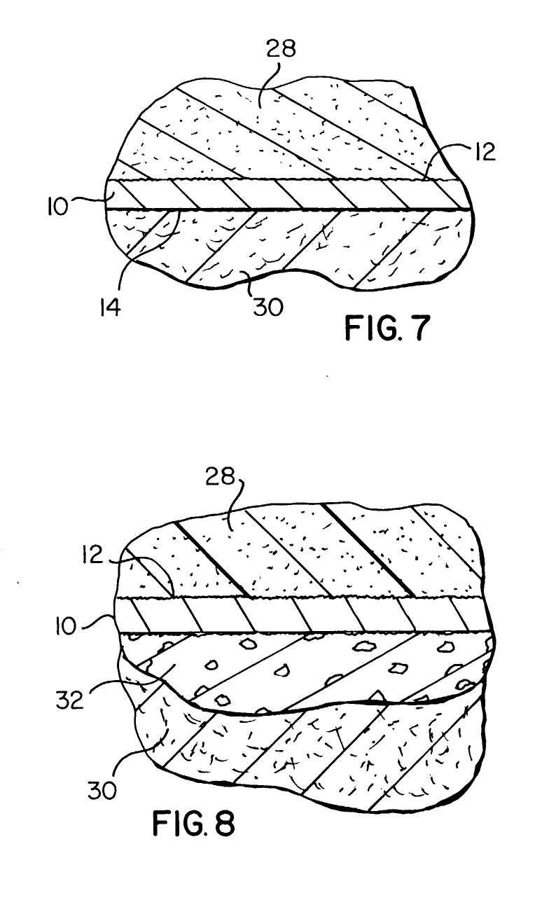

In FIGS. 7 and 8 are shown cross-sectional views along Line 7-7 and 8-8 respectively of FIG. 6.

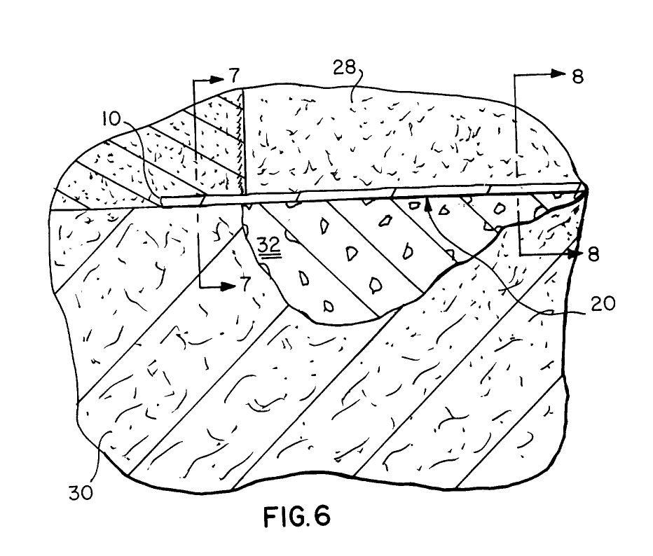

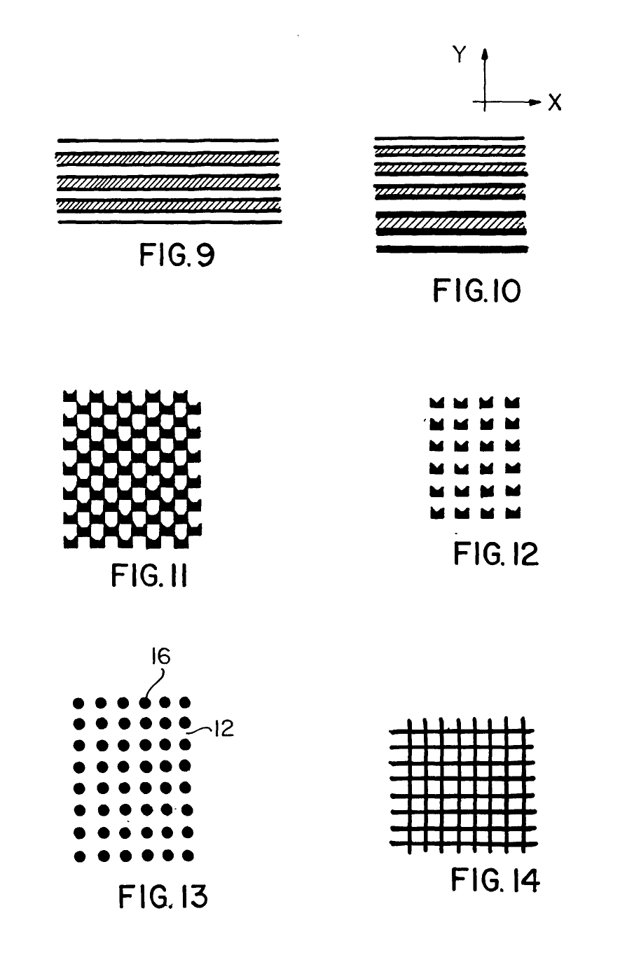

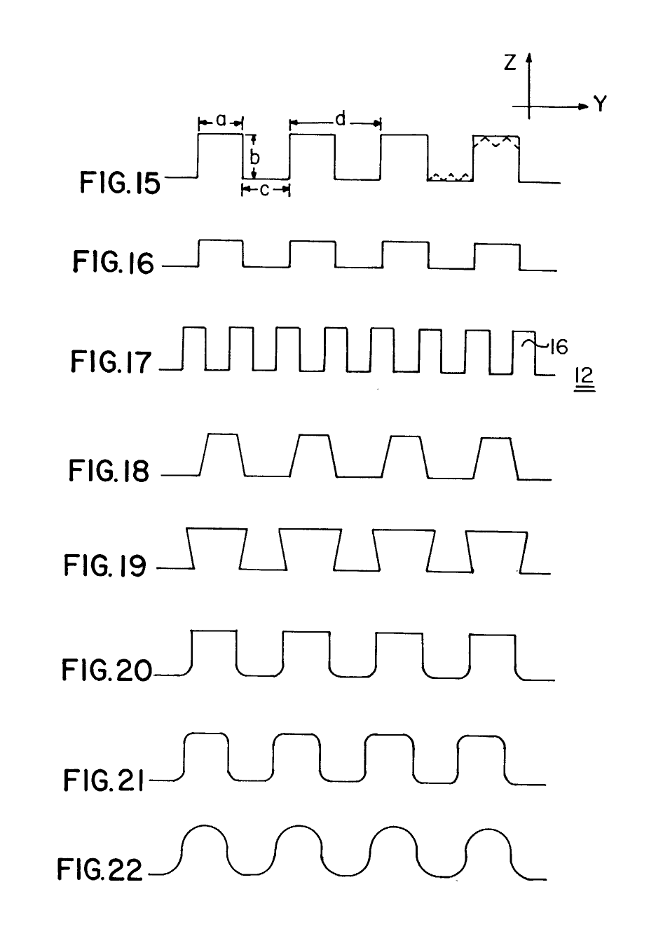

In FIGS. 9 to 14 are shown the range of potential surface geometries for either upper or lower surface 16 or 18, and in FIGS. 15 to 22 s shown a variety of vertical cross-sectional geometries the views of FIGS. 13 and 17 correspond to the views of FIGS. 3 and 5 above), each of these subject to the limitation that soft tissue will generally require a smaller scale of micro-texturzation than will that of bone tissue.

While there has been shown and described the preferred embodiment of the instant invention it is to be appreciated that the invention may be embodied otherwise than is herein specifically shown and described and that, within said embodiment, certain changes may be made in the form and arrangement of the parts without departing from the underlying ideas or principles of this invention as set forth in the claims appended herewith.

We claim:

1. An article for use in healing of wounds and repair of tissue defects, the article comprising:
(a) a flexible membrane having an upper and a lower surface, each surface defining a substrate formed of a biologically-acceptable biodegradable material adapted to be resorbed in use, each substrate having thereon means capable of orienting cell growth comprising a microgeometry formed in said substrates, a microgeometry of said upper surface proportioned to a cell morphology of soft tissue cells and a microgeometry of said lower surface proportioned to a cell morphology of bone tissue cells.

2. The article as recited in claim 1, in which said microgeometry of each substrate comprises a pattern of grooves and ridges.

3. The article as recited in claim 2, in which said ridges comprise posts.

4. The article as recited in claim 1, in which said membrane defines a width of between about 200 and about 500 microns.

5. The article as recited in claim 3, in which said grooves and ridges upon said upper surface defines a dimension of about 2 to about 10 microns, and those upon said lower surface define a dimension of about 8 to about 25 microns.

6. The article as recited in claim 5 in which said biodegradable material is selected from a member of the group consisting of as polylactic acid homopolymers, polyglycollic acid co-polymers, combinations thereof, polylactones, polypeptides, polyvinyl alcohols and natural polymers such as collagen and polysaccharides, collagen, Hench's bioglass, fibrinogen and polyimino-carbonate.

7. The article as recited in claim 1, in which a weight of said resorbed material is in a range of one to five grams/cm².

8. The article as recited in claim 3, in which said lower surface includes osteoconductive chemical properties.

9. The article as recited in claim 5, in which said lower surface includes osteoconductive chemical properties.

10. The article as recited in claim 5, in which said membrane comprises means for suturing into or about a wound or bone defect.
